(19) United States
(12) Patent Application Publication
(10) Pub. No.: US 2016/0278940 A1
(43) Pub. Date: Sep. 29, 2016

(54) INSTRUMENT SET FOR REMOVING A MODULAR CERAMIC HIP-JOINT IMPLANT

(71) Applicant: CERAMTEC GMBH, Plochingen (DE)

(72) Inventors: Marita Raschke, Leonberg (DE); Hendrik Bertmaring, Esslingen (DE); Kim Lars Häussler, Esslingen (DE); Sarah Richter, Ostfildern (DE)

(21) Appl. No.: 15/032,812

(22) PCT Filed: Oct. 31, 2014
(86) PCT No.: PCT/EP2014/073442

§ 371 Date: Apr. 28, 2016

(30) Foreign Application Priority Data
Oct. 31, 2013 (DE) 10 2013 222 161.4
Oct. 31, 2013 (DE) 10 2013 222 162.2

(51) Int. Cl. A61F 2/46; A61F 2/34

(52) U.S. Cl. CPC A61F 2/4609; A61F 2/34; A61F 2002/3409; A61F 2002/3482; A61F 2230/0095; A61F 2240/001; A61F 2230/0093

(57) ABSTRACT

An instrument set for removing a ceramic cup insert (I) from a metal shell (2) of a hip-joint implant has a holding element (3), a securing device (4) connectable to the holding element (3), at least one spacer (5) and a head (6). Holding element (3) has at one end an adapter device (7) for securing the head (6). Head (6) has an adapter location (8) for securing adapter device (7). Holding element (3) has at another end a coupling device (9) for a tool for rotating holding element (3) about its longitudinal axis (10). Holding element (3) has an adjusting thread (11) on its circumferential surface. Securing device (4) has an inner thread and can be turned on the adjusting thread (11) in the longitudinal axis (10) of the holding element (3). Spacer (5) is arranged on the securing device (4).

The invention relates to an instrument set for removing a ceramic cup insert from a metal shell of a hip joint implant.

Most ceramic hip joint implants are anchored in a modular fashion, by means of a conical clamping attachment, to a metal shell.

Reasons for possible explantation of the ceramic components might include:
Ceramic implant inserted at an angle;
Damaged ceramic implant (chip-off or crack, which could impair the function of the sliding surface, for example);
Damage or defects on the metal shell, which require a revision of the shell, and which can be carried out only with the ceramic removed.

The removal of the ceramic hip joint implant is presently only possible for specific systems using expensive supplementary instruments. In addition, intraoperative accesses do not allow the use of the supplementary instruments in each case (e.g. minimally invasive access with little “working space” for the surgeon).

Generally, the revision of a ceramic hip-joint implant is only rarely necessary. Therefore, hospitals often eschew the procurement of expensive revision systems. If no additional instruments are available at the hospital in question, the physician has to destroy the ceramic cup insert at great effort and with a high level of stress on the patient.

It is the object of the invention to develop an instrument set for removing a ceramic cup insert from a metal shell of a hip-joint implant, with which cost-effective and efficient intraoperative removal is made possible. Furthermore, the instrument set creates the possibility of a revision system for ceramic cup inserts.

According to the invention, this object is achieved with an instrument set according to the features of claim 1.

In that the instrument set for removing a ceramic cup insert from a metal shell of a hip joint implant, characterized in that the instrument set is composed of a holding element, a securing device connectable to the holding element, at least one spacer, and a head, wherein the holding element has, at one of its ends, an adapter device for securing the head, the head has an adapter location for securing the adapter device, the holding element having at its other end a coupling device for a tool for turning the holding element about its longitudinal axis, and the holding element has an adjusting thread on its circumferential surface, wherein the securing device is equipped with an inner thread, and can be turned on the adjusting thread in the longitudinal axis of the holding element, and at least one spacer is arranged on the securing device and extends parallel to the longitudinal axis of the holding element in the direction of the adapter device.

The instrument set according to the invention may be used for all metal shell sizes currently available on the market. Ideally, the instrument set can be used for metal shells with an outer diameter between 40 mm and 70 mm.

The instrument set has a head, preferably a spherical head, which is bonded in the ceramic cup insert of a hip joint implant. This head has a conformal shape to the shape of the cup insert. In a preferred embodiment, the head has a borehole at its adapting point with a thread. For removing the ceramic cup insert from a metal shell, the holding element according to the invention, which has an adapter device at one of its ends for securing the head, which, as a rule, consists of an adapter thread, is screwed onto the head. The head is also defined as disposable in a conical shape. The head can also be secured to the holding element with a bayonet closure or some other attachment device.

The instrument set should also be able to be pushed apart with the help of spacers at the edge of the metal shell. In order to ensure a uniform axial and thereby also patient-friendly procedure for removal, ideally three spacers should be preferred, each with a 120° offset. The spacers are ideally height-adjustable, in order to compensate for a possible angle offset. Alternatively, the metal shell can also be removed by a force impulse applied via an impact weight.

The instrument set should be made of surgical steel that is re-sterilizable, in order to assure repeated use and to avoid aging. The connections between the spacer and the metal shell should be arranged such that there is direct contact only between the metal shell and the instrument set, which continues during use.

Thanks to the bonded, conformal shape of the head, when using the instrument set ideally an axial traction is applied in order to remove the ceramic cup insert from the metal shell. The instrument set can be adapted for existing surgical instruments.

The underlying procedure, which is implemented with the instrument set, is described below.

Initially, by well-aimed blows to the edge of the metal shell, the interface strength between the ceramic, also called an insert, and the metal shell should be reduced.

Subsequently, the connection between the ceramic cup insert and a shape approximating the internal geometry of the ceramic cup insert, that is, the head, is produced. The connection may be form-fit, friction fit, or bonded. It is recommended that the connection be bonded using an adhesive. The connection surfaces should be adapted to the functional mode of the adhesive that is used (washed, degreased, and dried). Ideally, an adhesive on an acrylate basis should be used (for example: Histoacryl®) for the connection.

The bead should consist of a biocompatible material and offer the possibility of adapting to a surgical instrument (for example a sphere shaped from bone cement with an embedded surgical clamp). Ideally, the shape of the head is adapted to the sliding surface of the ceramic cup insert (sphere without cone but with thread) and consists of steel or plastic that can be used for the surgery, and can be reusable (re-sterilizable).

For removal, the holding element is rotated until the spacer is at the edge of the metal shell. Subsequently, the holding element is rotated further, by which an axial traction is built up on the cup insert, to the point where the head and cup insert connection is disengaged.

The adapter location of the head is therefore preferably a borehole with a thread. The adapter device on the threaded rod is preferably an adapter thread on the outer circumference of the threaded rod, which fits on the thread of the adapter location in the head. The pitch of the thread and possibly its multiple threading must be adjusted to the requirements.

The coupling device on the threaded rod is preferably a structure such as a flat, notch, or recess of the threaded rod. One preferable embodiment is suited for use of a box wrench.

In a preferred embodiment, there are three spacers with an offset of 120° on the holding element. However, a cup can also be used as a spacer, the edge of which must be supported on the edge of the metal cup.

In one embodiment, at the end of the spacer or spacers there is a support ring arranged with a support surface running perpendicular to the longitudinal axis of the threaded rod, whereby the support surface is pointing in the direction of the head. This support service must also be supported on the edge of the metal cup.

A hip joint implant according to the invention, with an outer metal shell in which a ceramic cup is anchored by means of a conical clamp, and the cup insert has a spherical recess for receiving the head, is—according to the invention—characterized in that the head is bonded in the spherical recess of the cup insert, and the head has an adapter location for securing part of the instrument set according to the invention for removing the cup insert from the metal shell.

A head as part of an instrument set according to the invention is characterized in that the bead is bonded in a spherical recess of the ceramic cup insert of a hip joint transplant, wherein the cup insert is connected via a conical clamp to a metal shell.

The use of an adhesive on an acrylate basis (for example: Histoacryl®) for bonding an above-described hip-joint implant or an above-described head is preferred.

The invention is described further below with reference to two figures.

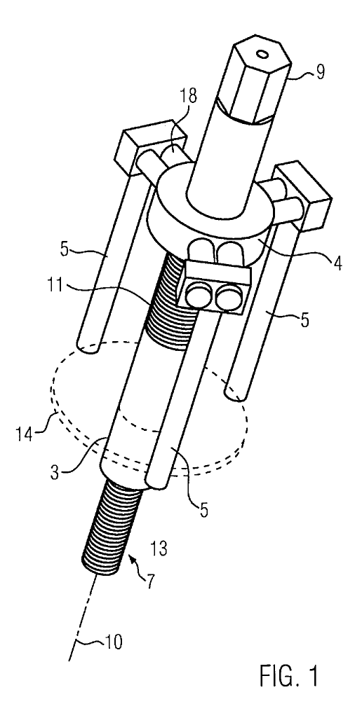

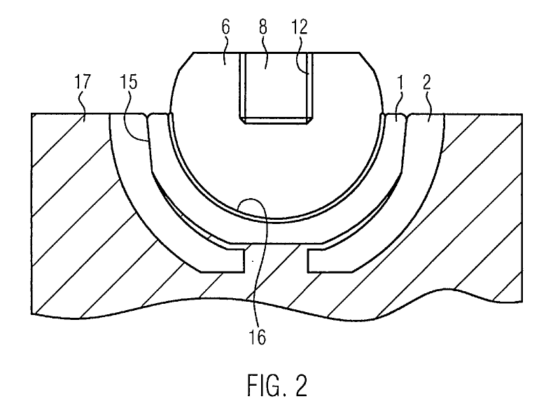

FIG. 1 up to the head 6 shows an instrument set according to the invention for removing a ceramic cup insert 1 from a metal shell 2 of a hip-joint implant. The metal shell is implanted in a basin 17 (see FIG. 2).

FIG. 2 shows in cross-section the basin 17 in which the metal shell 2 is implanted. A ceramic cup insert 1 is inserted in the metal shell and anchored by means of a conical clamp 15. It can happen that during an operation, the cup insert 1 has to be removed from the metal shell 2 again, for example in order to replace it. For this purpose, a head 6 as part of an instrument set according to the invention is placed in the spherical recess 16 of the cup insert 1 and bonded to the cup insert 1. The head 6 has a borehole 8 with a thread 12 on the part projecting out of the cup insert 2.

The term head is used because the cup insert 1 has a spherical recess 16. However, as FIG. 2 shows, the head 6 can also be hemispherical. The head 6 can also have a collar which lies on the edge of the metal shell 2. It is decisive that the head 6 is connected to the cup insert 6 in some way, preferably by bonding.

In a preferable embodiment, the head 6 is a spherical head. The holding elements 3 can be a threaded rod.

FIG. 1 shows the other parts of the instrument set. The central unit of the instrument set is a threaded rod 3, which at one end has an adapter device 7 for fastening to the head 6 (c.f. FIG. 2) and at its other end a coupling device 9. This coupling device 9 in the embodiment shown here is a hexagon. To turn the threaded rod 3, for example, a box wrench is placed on the coupling device 9 and thereby the holding element 3 is rotated about its longitudinal axis 10.

In the embodiment shown here, the adapter device 7 is an adapter thread 13 and the holding element 3 is screwed into the borehole 12 of the head 6 with its adapter thread 13.

An adjusting thread 11 is arranged between the adapter device 7 and the coupling device 9 on the threaded rod 3. On this adjusting thread 11 there is a holding element 4 with a thread which fits on the adjusting thread 4. On the holding element 4 are the spacers 5, arranged directly or as shown here via connecting elements 18, which extend in the direction of the adapter device 7. When the threaded rod 2 is turned, the holding element slides on the adjusting thread 11 as long as the spacers 5 lie on the metal shell 2 (see FIG. 2). Then, by further rotation of the threaded rod 3, the retaining strength of the cup insert 1 and the metal shell 2 is overcome. Once this occurs, the cup insert 1 can be raised out of the metal shell 2 with the described instrument set.

The instrument set may be used for all metal shell sizes currently available on the market. Ideally, the instrument set can be used for metal shells with an outer diameter between 40 mm and 70 mm.

The instrument set must have an adapter to which a conformal shape (at the adapter location, image 2) bonded to the hip-joint implant can be secured. Ideally, the conformal shape is connected by means of a thread or a rod to the instrument set.

Subsequently, it must be possible to push away the instrument set using spacers on the metal cup edge. In order to ensure a uniform and ideally axial and thereby also patient-sparing procedure for removal, ideally 3 spacers with 120° offsets should be used.

The instrument set should consist of surgical steel that is re-sterilizable in order to ensure multiple uses and to prevent aging. The connector points between the spacer and the metal shell should be arranged in such a way that there is direct contact only between the metal shell and the instrument set, which continues during use.

Thanks to the bonded, conformal shape, using the instrument set an axial traction should be applied in order to remove the ceramic cup insert from the metal shell. The instrument set should/can be adapted for existing surgical instruments.

The basic method that is to be implemented with the instrument set according to the invention is described as follows.

Initially, the interface strength between the ceramic insert and the metal shell should be reduced by well-aimed blows on the edge of the metal cop.

Subsequently, a connection should be established between the ceramic insert and a shape conforming to the inner geometry of the ceramic cup insert. The connection may be form-fit, friction fit, or bonded.

Initially, the connecting surfaces must be cleaned, degreased, and dried. It is recommended that the connection be bonded using an adhesive. In the best case an adhesive based on acrylate (for example: Histoacryl®) is used.

The connected form should consist of biocompatible material and offer the possibility of adapting for a surgical instrument (for example a clamp in bone cement or impactor ball with thread). Ideally, the shape is adapted to the sliding surface of the ceramic (sphere without cone but with threading), consists of a steel or plastic possible for surgical use, and is reusable (re-sterilizable).

1.-10. (canceled)

11. An instrument set for removing a ceramic cup insert from a metal shell of a hip-joint implant, wherein the instrument set comprises
a holding element;
a securing device connectable to the holding element;
at least one spacer; and
a head;
wherein the holding element has at one end thereof an adapter device for securing the head;
wherein the head has an adapter location for securing the adapter device;
wherein the holding element has at another end thereof a coupling device for a tool for turning the holding element about its longitudinal axis;
wherein the holding element has an adjusting thread on its circumferential surface;
wherein the securing device comprises an inner thread, and can be turned on the adjusting thread in the longitudinal axis of the holding element; and
wherein the at least one spacer is arranged on the securing device and extends parallel to the longitudinal axis of the holding element in the direction of the adapter device.

12. The instrument set according to claim 11, wherein the adapter location of the head is a borehole with a thread or a bayonet closure.

13. The instrument set according to claim 11, wherein the adapter device is an adapter thread on the outer circumference of the holding element, which fits with the thread of the adapter location in the head.

14. The instrument set according to claim 11, wherein the coupling device comprises at least one structure selected from the group consisting of a flat, a notch, or a recess on the outer circumference of the holding element.

15. The instrument set according to claim 11, wherein three spacers are arranged on the securing device with a 120° offset between them.

16. The instrument set according to claim 11, wherein at the end of the spacer or spacers, a support ring is arranged with a support surface running perpendicularly to the longitudinal axis of the holding element, wherein the supporting surface points in the direction of the head.

17. A hip-joint implant with an outer metal shell in which a ceramic cup insert is anchored by means of a conical clamp and the cup insert has a spherical recess for receiving a head, wherein the head is bonded in the spherical recess of the cup insert and the head has an adapter location for securing a part of an instrument set for removing the cup insert from the metal shell in accordance with claim 11.

18. A head as part of an instrument set according to claim 11, wherein the head is adhered in a spherical recess of a ceramic cup insert of a hip-joint implant, wherein the cup insert is connected via a conical clamp to a metal shell.

19. A method comprising applying an adhesive comprising an acrylate to bond the hip-joint implant in accordance with claim 17.

20. A method comprising applying an adhesive comprising an acrylate to bond the head in accordance with claim 18.

21. An instrument set according to claim 11, wherein the head has a shape that conforms to the spherical recess in the cup insert.

22. A hip-joint implant according to claim 17, wherein the head has a shape that conforms to the spherical recess in the cup insert.

23. A head according to claim 18, wherein the head has a shape that conforms to the spherical recess in the cup insert.

24. The instrument set according to claim 12, wherein the adapter device is an adapter thread on the outer circumference of the holding element, which fits with the thread of the adapter location in the head.

25. The instrument set according to claim 12, wherein the coupling device comprises at least one structure selected from the group consisting of a flat, a notch, or a recess on the outer circumference of the holding element.

26. The instrument set according to claim 13, wherein the coupling device comprises at least one structure selected from the group consisting of a flat, a notch, or a recess on the outer circumference of the holding element.

27. The instrument set according to claim 12, wherein three spacers are arranged on the securing device with a 120° offset between them.

28. The instrument set according to claim 13, wherein three spacers are arranged on the securing device with a 120° offset between them.

29. The instrument set according to claim 14, wherein three spacers are arranged on the securing device with a 120° offset between them.

30. The instrument set according to claim 12, wherein at the end of the spacer or spacers, a support ring is arranged with a support surface running perpendicularly to the longitudinal axis of the holding element, wherein the supporting surface points in the direction of the head.
